(19) United States
(12) Patent Application Publication
(10) Pub. No.: US 2025/0345136 A1
(43) Pub. Date: Nov. 13, 2025

(54) HANDPIECE TRIGGERING DEVICE

(71) Applicants: Smith & Nephew, Inc., Memphis, TN (US); Smith & Nephew Orthopaedics AG, Zug (CH)

(72) Inventors: Ryan Sheehan, Pittsburgh, PA (US); Branislav Jaramaz, Pittsburgh, PA (US); Constantinos Nikou, Monroeville, PA (US)

(73) Assignees: Smith & Nephew, Inc., Memphis, TN (US); Smith & Nephew Orthopaedics AG, Zug (CH)

(21) Appl. No.: 19/192,959

(22) Filed: Apr. 29, 2025

Related U.S. Application Data
(60) Provisional application No. 63/644,671, filed on May 9, 2024.

(51) Int. Cl. A61B 34/35; A61B 17/00; A61B 17/16; A61B 90/00

(52) U.S. Cl. CPC A61B 34/35; A61B 17/1675; A61B 2017/00221; A61B 2017/00367; A61B 2090/064

(57) ABSTRACT

A system and a method for controlling a surgical system. The system includes a wireless actuator, which upon actuation, is configured to generate and transmit one or more actuation instructions. The system further includes a control processor communicatively coupled to the wireless actuator, where the control processor is configured to receive one or more actuation instructions from the wireless actuator, generate, using one or more actuation instructions, one or more communication instructions for triggering actuation of at least one operation of one or more surgical components of a surgical system communicatively coupled to the control processor, and transmit, via an antenna communicatively coupled to the control processor, one or more communication instructions to the surgical system.

### CROSS-REFERENCE TO RELATED APPLICATIONS

This is a non-provisional of, and claims the benefit of the filing date of, pending U.S. Provisional Patent Application No. 63/644,671, filed May 9, 2024, entitled “Handpiece Triggering Device,” the entirety of which application is incorporated by reference herein.

### BACKGROUND

Robotically assisted surgeries, such as, for example, a total knee arthroplasty (TKA), provide a surgeon with an advantage of planning a procedure and viewing a projected outcome of the procedure prior to performing bone resection. One of the challenges to providing a robotically assisted surgery is optimizing control operation of various surgical hardware components (e.g., a handpiece, etc.) of the surgical system being used to perform such surgery. Conventionally, such components are controlled using a foot pedal that a surgeon can press with their foot to activate and/or deactivate a particular hardware component. The foot pedal may be connected to the surgical system using one or more wires and may include one or more pressable buttons that the surgeon may step on or press (e.g., with their foot) to activate various functions of the hardware component. In the case of a handpiece, such buttons may be pressed to execute variable speed control operation of the cutting tool.

However, a foot pedal, let alone any of its buttons, might not be clearly visible to the surgeon who is typically more focused on the surgical site. Such lack of visibility of the foot pedal and its buttons can cause the surgeon to press a wrong button triggering unwanted operation (and/or cessation thereof) of the handpiece.

Moreover, cables connecting the foot pedal to the surgical system may cause the surgeon to trip and fall resulting in physical injuries and/or other undesired consequences to the patient and/or the surgeon. Thus, it may be advantageous to control the surgical hardware (and/or software) components using hand-based movements, e.g., pressing a button, tapping or applying pressure to a pad, etc. However, in conventional systems, it is nearly impossible to alter physical hardware and/or software configuration (no matter how small) of a surgical system to implement such hand-based control functionalities, as, such changes, typically require not only reconfiguring wiring, hardware, and/or software aspects of the surgical system, but also design of operation protocols, testing, experimentation, data collection, verification, etc. and regulatory approval. These can significantly delay and/or prevent implementation of the surgical system, thereby denying vital medical care to patients.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to a system that may include a wireless actuator that may, upon actuation, be configured to generate and transmit one or more actuation instructions. The system may also include a control processor communicatively coupled to the wireless actuator. The control processor may be configured to receive one or more actuation instructions from the wireless actuator, generate, using one or more actuation instructions, one or more communication instructions for triggering actuation of at least one operation of one or more surgical components of a surgical system communicatively coupled to the control processor, and transmit, via an antenna communicatively coupled to the control processor, one or more communication instructions to the surgical system.

In any preceding or subsequent examples, upon a first actuation, the wireless actuator may be configured to generate and transmit a first actuation instruction to the control processor causing the control processor to generate and transmit a first communication instruction triggering actuation of a first operation of one or more surgical components. Upon a second actuation, the wireless actuator may be configured to generate and transmit a second actuation instruction to the control processor causing the control processor to generate and transmit a second communication instruction triggering actuation of a second operation of one or more surgical components. The second operation may be subsequent to the first operation.

In any preceding or subsequent examples, the second operation may be different from the first operation. Alternatively, or in addition, the second operation may be the same as the first operation. In any preceding or subsequent examples, the first operation may be a surgical cutting operation performed by one or more components of the surgical system at a first speed and a second operation may be a surgical cutting operation performed by one or more components of the surgical system at a second speed. In any preceding or subsequent examples, the second speed may be faster, same or slower than the first speed. In any preceding or subsequent examples, at least one of the first speed and the second speed may be at least one of: a variable speed, a constant speed, and any combination thereof.

In any preceding or subsequent examples, the wireless actuator may include a momentary push button and a trigger. Upon pressing the trigger, the trigger may be configured to apply pressure to the momentary push button causing the momentary push button to generate and transmit one or more actuation instructions.

In any preceding or subsequent examples, the wireless actuator may include a linear potentiometer and a trigger. Upon pressing the trigger, the linear potentiometer may be configured to determine its linear displacement causing the linear potentiometer to generate and transmit one or more actuation instructions.

In any preceding or subsequent examples, the wireless actuator may include a pressure sensor and a trigger. Upon pressing the trigger, the pressure sensor may be configured to detect a predetermined force applied to it by the trigger causing the pressure sensor to generate and transmit one or more actuation instructions.

In any preceding or subsequent examples, the wireless actuator may include a pressure sensor. Upon applying a force to the pressure sensor, the pressure sensor may be configured to detect the force causing the pressure sensor to generate and transmit one or more actuation instructions.

In any preceding or subsequent examples, the control processor may include a sensing circuit configured to receive one or more actuation instructions, interpret one or more actuation instructions, and generate and transmit, via the antenna communicatively coupled to the control processor, one or more communication instructions to the surgical system.

In any preceding or subsequent examples, at least one of the control processor, the antenna, and the wireless actuator may be disposed in one or more surgical components of the surgical system.

In some examples, the present disclosure relates to a method. The method may include receiving, using at least one processor, one or more actuation instructions from a wireless actuator communicatively coupled to the processor, where the wireless actuator, upon actuation, may be configured to generate and transmit one or more actuation instructions to the processor, generating, using one or more actuation instructions, one or more communication instructions for triggering actuation of at least one operation of one or more surgical components of a surgical system communicatively coupled to the processor, and transmitting, via an antenna communicatively coupled to the processor, one or more communication instructions to the surgical system.

In any preceding or subsequent examples, upon a first actuation, the wireless actuator is configured to generate and transmit a first actuation instruction to the processor causing the processor to generate and transmit a first communication instruction triggering actuation of a first operation of one or more surgical components. Upon a second actuation, the wireless actuator is configured to generate and transmit a second actuation instruction to the processor causing the processor to generate and transmit a second communication instruction triggering actuation of a second operation of one or more surgical components. The second operation may be subsequent to the first operation. The first operation may be a surgical cutting operation performed by one or more components of the surgical system at a first speed and a second operation may be a surgical cutting operation performed by one or more components of the surgical system at a second speed.

Examples of the present disclosure provide numerous advantages. For example, the current subject matter's use of wireless communication components and microprocessors enables seamless integration of control mechanisms for controlling operation of a surgical system without materially or substantially altering hardware and/or software configuration of such surgical system. Moreover, wireless communication components and microprocessors remove the need to incorporate bulky cabling systems for controlling operation of the surgical system, thereby preventing accidents, errors, and/or any other malfeasance in the operating room. Additionally, one or more components (e.g., the processor, the wireless actuator, etc.) of the current subject matter system may be disposable making it easier to replace when they malfunction, or an upgrade is needed, and/or they (and/or any other components of the surgical system) need to be sterilized, all of which are difficult to do in conventional systems.

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings,

FIG. 1 illustrates an example surgical system for performing a surgical procedure using a robotic system;

FIG. 2 is a block diagram depicting an example system for performing a robotically assisted surgical procedure;

FIG. 3A illustrates an example system that uses a momentary push button for controlling of one or more components of a surgical system, according to some examples of the current subject matter;

FIG. 3B illustrates an example system that uses a linear potentiometer for controlling of one or more components of a surgical system, according to some examples of the current subject matter;

FIG. 3C illustrates an example system that uses a pressure sensor for controlling of one or more components of a surgical system, according to some examples of the current subject matter;

FIG. 3D illustrates an example system that uses a pressure sensor for controlling of one or more components of a surgical system, according to some examples of the current subject matter.

FIG. 4A illustrates an example surgical system, according to some examples of the current subject matter;

FIG. 4B illustrates another example surgical system, according to some examples of the current subject matter;

FIG. 5 illustrates a process for controlling operation of the surgical cutting tool, according to some examples of the current subject matter;

FIG. 6 illustrates another process for controlling operation of the surgical cutting tool, according to some examples of the current subject matter;

FIG. 7 illustrates an example of a storage medium to store logic in accordance with one or more features of the present disclosure; and

FIG. 8 illustrates an example computing platform in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide a system and a method for controlling operation of a surgical system.

In some examples, the current subject matter provides a wireless hand-based control system that may assist a surgeon in controlling one or more operations and/or actuation of one or more operation of a surgical system, such as, for example, but not limited to a surgical system for performing a TKA. The system may be used to control actuation and/or operation of a surgical cutting tool used to the TKA. One or more components the current subject matter system may be disposable, thereby making it easier to replace and/or substitute, such as, for example, when one or more of such components malfunction, an upgrade is needed, and/or sterilization of such components and/or any other components of the surgical system is needed, etc.

The system may include a wireless actuator and a control processor. The wireless actuator may be communicatively coupled to the control processor via a wireless communication connection. For example, the wireless actuator and the control processor may be communicatively coupled via any wireless communication protocols, e.g., WiFi, Bluetooth, IOlink, Zigbee, cellular (e.g., 4G LTE, 4G LTE-A, 5G, etc.), and/or any other desired protocol (whether proprietary or not). In some examples, one or more custom wireless communication component(s) (e.g., implementing its own custom-designed software, firmware, hardware, etc.) may be used by the wireless actuator and/or the control processor and/or both to communicate with one another, where the custom wireless communication component(s) may implement one or more known wireless communication protocols and/or one or more proprietary communication protocols that may be designed for use with the custom wireless communication component(s). The custom wireless communication component(s) may include, for example, one or more receiver(s), one or more transmitter(s) and/or one or more transceiver(s) that may be communicatively coupled to and/or be integrated with the wireless actuator and/or the control processor. Use of such custom wireless communication component(s) and/or proprietary wireless communication protocol(s) may allow for a greater control, security, lower latency, etc. of wireless communications between the wireless actuator and the control processor. Upon actuation, the wireless actuator may be configured to generate and transmit one or more actuation instructions, which may be represented by one or more signals. The wireless actuator may include processing circuitry and a transmission circuitry (e.g., an antenna, a wireless transceiver, etc.) that may be used generation of such actuation instructions.

In some examples, the wireless actuator may include a momentary push button and a trigger. To actuate the wireless actuator (thereby causing to generate and transmit actuation instructions), the trigger may be pressed (e.g., by a finger, a hand, a foot, etc. of the surgeon performing the surgery), which may cause it to apply pressure to the momentary push button. Once the push button detects pressure applied to it by the trigger, the push button circuitry may be configured to generate the actuation instructions and transmit them to the control processor. In some examples, the control processor may include a sensing circuit. The sensing circuit may include various hardware and/or software components, including, but not limited to, an antenna, a transceiver, etc. Once actuation instructions from the push button are received, the sensing circuit of the control processor may be configured interpret the instructions and generate surgical system component operation communication instructions for transmission to the surgical system. The control processor may transmit the communication instructions to the surgical system via an antenna that may be communicatively coupled to the control processor. In some examples, at least one of the control processor, the antenna, and the wireless actuator may be disposed in one or more surgical components (e.g., a cutting tool) of the surgical system. Alternatively, or in addition, at least one of the control processor, the antenna, and the wireless actuator may be disposed separately from one or more surgical components of the surgical system. As can be understood, any desired variations of disposing at least one of the control processor, the antenna, and the wireless actuator are possible.

In alternate examples, the wireless actuator may include a linear potentiometer and a trigger. A linear potentiometer is a type of position sensor, which may be used to measure a displacement along a single axis (e.g., up and down, left and right, etc.). A typical linear potentiometer may be rod actuated and may be connected to an internal slider, a wiper carrier, etc. In this example, the trigger may be pressed, thereby applying pressure to the linear potentiometer. Upon detecting pressure, the rod in the linear potentiometer may be configured to be linearly displaced. Once a predetermined linear displacement is detected by the linear potentiometer circuitry, the linear potentiometer may be configured to generate and transmit one or more actuation instructions (similar to the momentary push button example discussed above).

In yet further alternate examples, the wireless actuator may include a pressure sensor and a trigger. A pressure sensor may be a transducer that may convert an input mechanical pressure into an electrical output signal (e.g., pressure sensor defined), where the output electrical system may be used to generate actuation instructions by the pressure sensor circuitry. There are several types of pressure sensors, where use of each may be based on size, capacity, measurement method, sensing technology, output requirements, etc. In this example, the trigger may likewise be pressed, thereby applying pressure to the pressure sensor. Upon detecting a predetermined pressure, the pressure sensor may be configured to generate and transmit one or more actuation instructions (similar to the momentary push button and linear potentiometer examples discussed above). Alternatively, or in addition, the pressure sensor may be used without the trigger, where pressure may be directly applied by the surgeon performing the surgery.

In some examples, each actuation of the wireless actuator may be configured to trigger actuation of a different action by one or more surgical components of the surgical system. For example, one actuation (e.g., a press of the trigger on the momentary push button) may trigger operation of the cutting instrument at a first speed. Another actuation (e.g., another press (e.g., a double press) of the trigger on the momentary push button) may trigger operation of the cutting instrument at a second speed. The first and second speeds may be same and/or different (e.g., slower, faster, and/or the same). In some non-limiting examples, the speeds may be variable and/or constant. Each actuation of the actuator may be configured to cause generation and transmission of its own corresponding actuation instruction(s) to the control processor, which may cause the control processor to generate and transmit respective corresponding communication instruction(s) to the surgical component(s) of the surgical system.

As can be understood, one or more wireless actuators may be used by the current subject matter system. For instance, one wireless actuator may be used to control operation of the cutting instrument, another wireless actuator may be used to control operation of optical tracking system of the surgical system, etc. The actuators may be the same and/or different (e.g., one actuator is a momentary push button and another actuator is a linear potentiometer). A single wireless actuator may be used to control one or more operations of one or more components of the surgical system (e.g., cutting instruments, optical tracking system, graphical user interface(s), etc.). A single wireless actuator may incorporate multiple types of actuation mechanisms (e.g., momentary push button(s), linear potentiometer(s), etc.), where each actuation mechanism may control separate (or same) components of the surgical system. As can be understood, one or more actuators may be wired actuators.

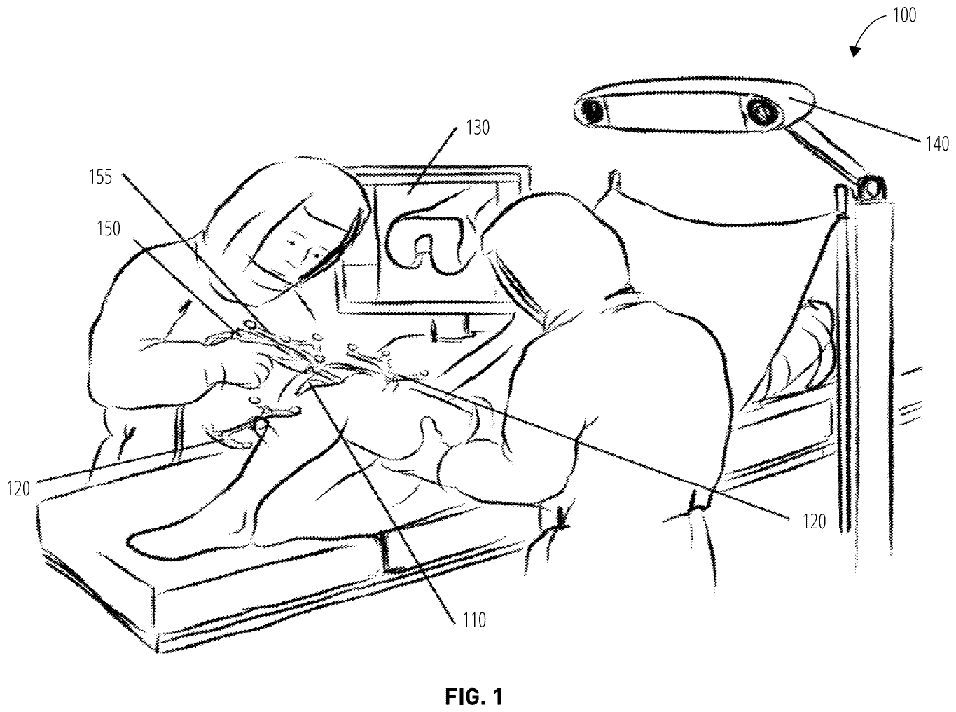

FIG. 1 illustrates an example surgical system 100 for performing a surgical procedure using a robotic system. The system 100 may incorporate the current subject matter system for controlling operation of one or more components of the surgical system 100.

The surgical system 100 can include a surgical cutting tool 150 along with an associated optical tracking frame 155 (also referred to as a tracking array 155), a display device 130, an optical tracking system 140, and one or more patient tracking frames 120 (also referred to as tracking arrays 120). The system 100 can be used by one or more medical professionals, e.g., surgeons, to perform a surgery, such as, for example, the TKA. The surgery can be performed by making an incision 110 in the knee of a patient and conducting further steps of the knee replacement surgery. As can be understood, the system 100 may be used for performance of any desired surgical procedures. The knee replacement surgery is used herein as an illustrative example and is not intended to limit the subject matter disclosed herein.

The surgical system 100 can include a hand-held, computer-controlled surgical robotic system that uses the optical tracking system 140 coupled to the robotic controller to track and control a hand-held surgical instrument. For example, the optical tracking system 140 tracks the tracking array 155 coupled to the surgical tool 150 and tracking arrays 120 coupled to the patient to track a location of the instrument relative to the target bone (e.g., femur and tibia for knee procedures).

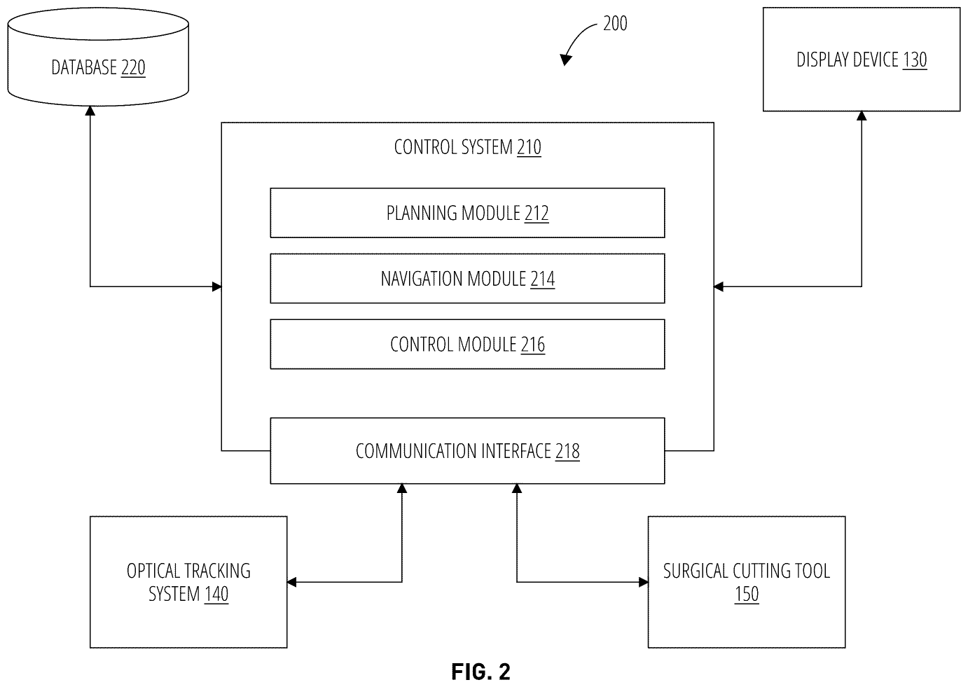

FIG. 2 is a block diagram depicting an example system 200 for performing a robotically assisted surgical procedure. The system 200 can be incorporated into the system 100 shown in FIG. 1 and used by the current subject matter system to control operation of one or more surgical components (e.g., surgical cutting tool 150).

In some examples, the system 200 can include a control system 210, the optical tracking system 140, and the surgical cutting tool 150. Optionally, the system 200 can include a display device 130 and the database 220. In some examples, these components can be combined to provide navigation and control of the surgical cutting tool 150, which can include navigation and control of a cutting tool 150 and/or a point probe, among other things, which can be used during an orthopedic surgery (and/or any other surgery).

One or more components of the system shown in FIG. 2 may be communicatively coupled using one or more communications networks. The communications networks may include one or more of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof.

Further, one or more components of the system shown in FIG. 2 may include any combination of hardware and/or software. In some examples, one or more components of the system may be disposed on one or more computing devices, such as, server(s), database(s), personal computer(s), laptop(s), cellular telephone(s), smartphone(s), tablet computer(s), virtual reality devices, and/or any other computing devices and/or any combination thereof. In some examples, one or more components of the system may be disposed on a single computing device and/or may be part of a single communications network. Alternatively, or in addition to, such devices may be separately located from one another. A device may be a computing processor, a memory, a software functionality, a routine, a procedure, a call, and/or any combination thereof that may be configured to execute a particular function associated with interface and/or document certification processes disclosed herein.

In some examples, one or more components of the system shown in FIG. 2 may include network-enabled computers. As referred to herein, a network-enabled computer may include, but is not limited to a computer device, or communications device including, e.g., a server, a network appliance, a personal computer, a workstation, a phone, a smartphone, a handheld PC, a personal digital assistant, a thin client, a fat client, an Internet browser, or other device. One or more components of the system also may be mobile computing devices, for example, an iPhone, iPod, iPad from Apple® and/or any other suitable device running Apple's iOS® operating system, any device running Microsoft's Windows®. Mobile operating system, any device running Google's Android® operating system, and/or any other suitable mobile computing device, such as a smartphone, a tablet, or like wearable mobile device.

One or more components of the system shown in FIG. 2 may include a processor and a memory, and it is understood that the processing circuitry may contain additional components, including processors, memories, error and parity/CRC checkers, data encoders, anti-collision algorithms, controllers, command decoders, security primitives and tamper-proofing hardware, as necessary to perform the interface and/or document certification functions described herein. One or more components of the system may further include one or more displays and/or one or more input devices. The displays may be any type of devices for presenting visual information such as a computer monitor, a flat panel display, and a mobile device screen, including liquid crystal displays, light-emitting diode displays, plasma panels, and cathode ray tube displays. The input devices may include any device for entering information into the user's device that is available and supported by the user's device, such as a touchscreen, keyboard, mouse, cursor-control device, touchscreen, microphone, digital camera, video recorder or camcorder. These devices may be used to enter information and interact with the software and other devices described herein.

In some examples, one or more components of the system shown in FIG. 2 may execute one or more applications, such as software applications, that enable, for instance, network communications with one or more components of system and transmit and/or receive data.

One or more components of the system shown in FIG. 2 may include and/or be in communication with one or more servers via one or more networks and may operate as a respective front-end to back-end pair with one or more servers. One or more components of the system may transmit, for example from a mobile device application (e.g., executing on one or more user devices, components, etc.), one or more requests to one or more servers. The requests may be associated with retrieving data from servers. The servers may receive the requests from the components of the system. Based on the requests, servers may be configured to retrieve the requested data from one or more storage locations. Based on receipt of the requested data from the databases, the servers may be configured to transmit the received data to one or more components of the system, where the received data may be responsive to one or more requests.

The system shown in FIG. 2 may include one or more networks. In some examples, networks may be one or more of a wireless network, a wired network or any combination of wireless network and wired network and may be configured to connect the components of the system and/or the components of the system to one or more servers. For example, the networks may include one or more of a fiber optics network, a passive optical network, a cable network, an Internet network, a satellite network, a wireless local area network (LAN), a metropolitan area network (MAN), a wide area network (WAN), a virtual local area network (VLAN), an extranet, an intranet, a Global System for Mobile Communication, a Personal Communication Service, a Personal Area Network, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or any other type of network and/or any combination thereof.

In addition, the networks may include, without limitation, telephone lines, fiber optics, IEEE Ethernet 802.3, a wide area network, a wireless personal area network, a LAN, or a global network such as the Internet. Further, the networks may support an Internet network, a wireless communication network, a cellular network, or the like, or any combination thereof. The networks may further include one network, or any number of the exemplary types of networks mentioned above, operating as a stand-alone network or in cooperation with each other. The networks may utilize one or more protocols of one or more network elements to which they are communicatively coupled. The networks may translate to or from other protocols to one or more protocols of network devices. The networks may include a plurality of interconnected networks, such as, for example, the Internet, a service provider's network, a cable television network, corporate networks, such as credit card association networks, and home networks.

The system shown in FIG. 2 may include one or more servers, which may include one or more processors that may be coupled to memory. Servers may be configured as a central system, server or platform to control and call various data at different times to execute a plurality of workflow actions. Servers may be configured to connect to the one or more databases. Servers may be incorporated into and/or communicatively coupled to at least one of the components of the system.

Further, one or more components of the system shown in FIG. 2 may be configured to execute one or more actions using one or more containers. In some examples, each action may be executed using its own container. A container may refer to a standard unit of software that may be configured to include the code that may be needed to execute the action along with all its dependencies. This may allow execution of actions to run quickly and reliably.

The control system 210 can include one or more computing devices configured to coordinate information received from the optical tracking system 140 and provide control to the surgical cutting tool 150. In some examples, the control system 210 can include a planning module 212, a navigation module 214, a control module 216, and a communication interface 218. The planning module 212 can provide pre-operative planning capabilities that allow surgeons to virtually plan a procedure prior to reshaping a target joint during the surgical procedure on the patient.

In some examples, the planning module 212 can be used to manipulate a virtual model of the implant in reference to a virtual implant host model (such as, for instance, for the purposes of the TKA). The virtual model of the implant host (illustrating the joint to be replaced) can be created through use of a point probe or similar instrument tracked by the optical tracking system 140. The planning module 212 can collect data from surfaces of the target joint to recreate a virtual model of the patient's actual anatomical structure. By way of a non-limiting example, in a joint replacement surgery, this can increase accuracy of the planning process by using data collected after the joint has been exposed and without intra-operative imaging. Collecting surface data from the target bone(s) also can allow for iterative reshaping of the target bone to ensure proper fit of the prosthetic implants and optimization of anatomical alignment.

In some examples, the navigation module 214 can coordinate tracking the location and orientation of the implant, the implant host, and the surgical cutting tool 150 during the surgical procedure. Further, the navigation module 214 can also coordinate tracking of the virtual models used during pre-operative or intra-operative planning within the planning module 212. Tracking the virtual models can include operations such as alignment of the virtual models with the implant host through data obtained via the optical tracking system 140. The navigation module 214 can receive input from the optical tracking system 140 regarding the physical location and orientation of the surgical cutting tool 150 and an implant host. Tracking of the implant host can include tracking multiple individual bone structures, such as with patient tracking frames 120. For example, during a total knee replacement procedure, the optical tracking system 140 can individually track the femur and the tibia using tracking devices anchored to the individual bones (as shown, for example, in FIG. 1).

In some examples, the control module 216 can process information provided by the navigation module 214 to generate control signals for controlling the surgical cutting tool 150. The control module 216 also can work with the navigation module 214 to produce visual animations to assist the surgeon during an operative procedure. Visual animations can be displayed via a display device, such as, for instance, display device 130. In some examples, the visual animations can include real-time 3D representations of the implant, the implant host, and the surgical cutting tool 150, among other things. Further, the visual animations can be color-coded to further assist the surgeon with positioning and orienting the implant.

The communication interface 218 can facilitate communication between the control system 210 and one or more external systems and/or devices. The communication interface 218 can include wired and/or wireless communication interfaces, such as Ethernet, IEEE 802.11 wireless, or Bluetooth, among others. As illustrated in FIG. 1, the primary external systems connected via the communication interface 218 can include the tracking system 140 and the surgical instrument 150. Although not shown, the database 230 and the display device 130, among other devices, also can be connected to the control system 210 via the communication interface 218. In some examples, the communication interface 218 can communicate over an internal bus to other modules and hardware systems within the control system 210.

The optical tracking system 140 can provide location and orientation information for surgical devices and parts of an implant host's anatomy to assist in navigation and control of semi-active robotic surgical devices. The optical tracking system 140 can include a tracker (e.g., patient tracking frames 120) that can include and/or otherwise provide tracking data based on one or more (e.g., three) positions and/or one or more (e.g., three) angles. The tracker can include one or more first tracking markers associated with the implant host and one or more second markers associated with the surgical device (e.g., surgical cutting tool 150). The markers and/or some of the markers can be one or more of infrared sources, light emitting sources, radio frequency (RF) sources, ultrasound sources, and/or transmitters. The optical tracking system 140 can be an infrared tracking system, an optical tracking system, an ultrasound tracking system, an inertial tracking system, a wired system, an RF tracking system, and/or any other type of system and/or any combination thereof.

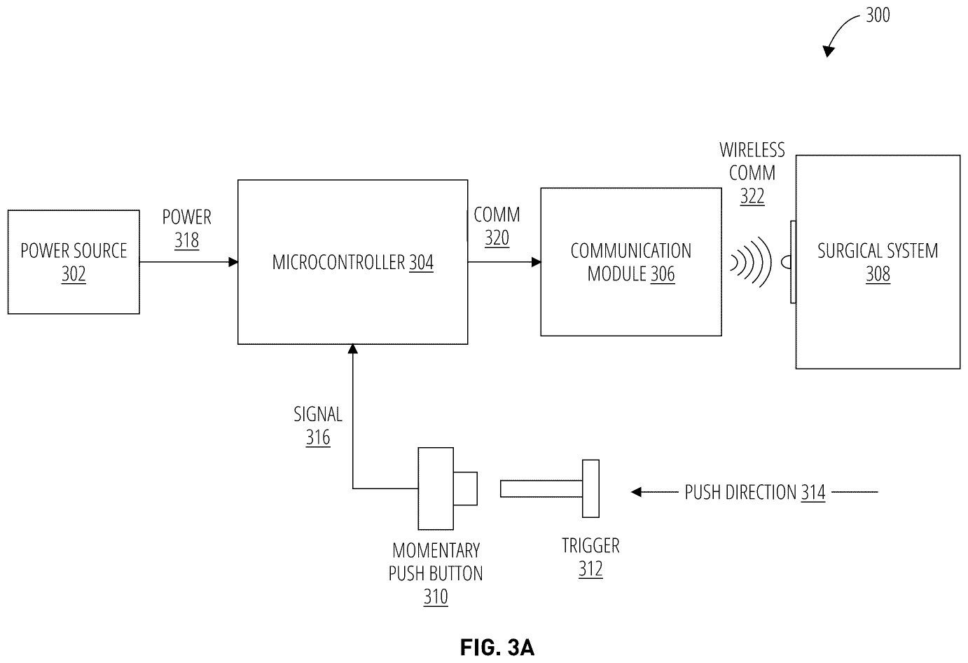

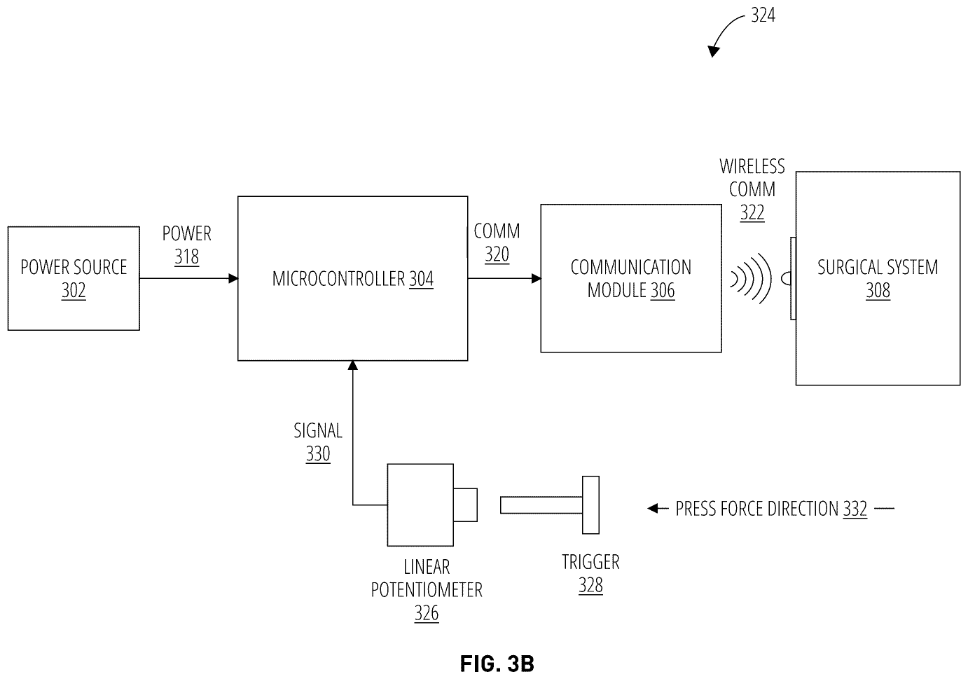

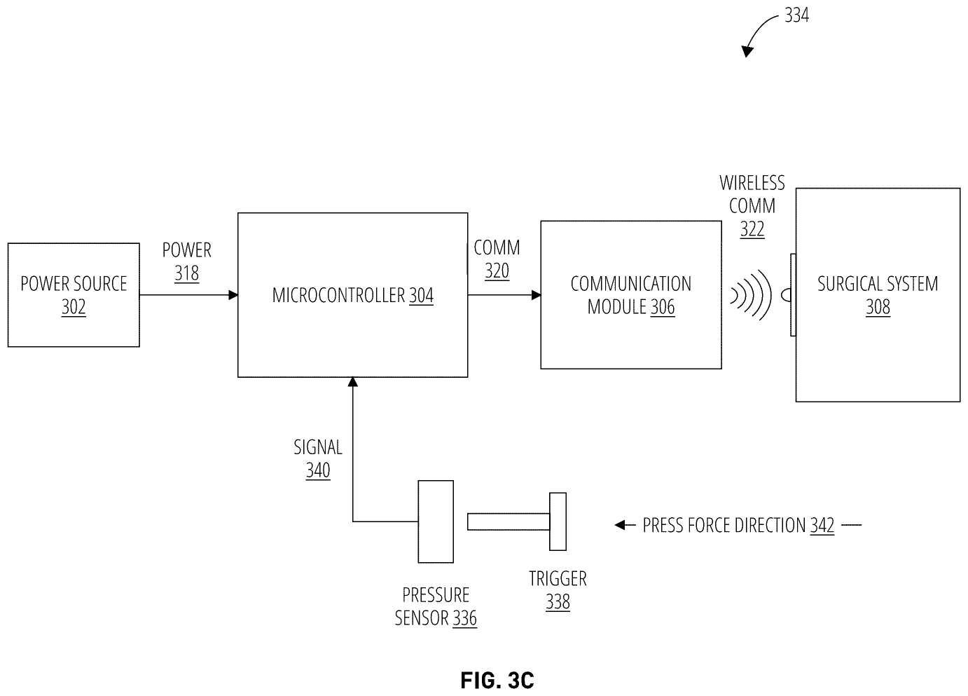

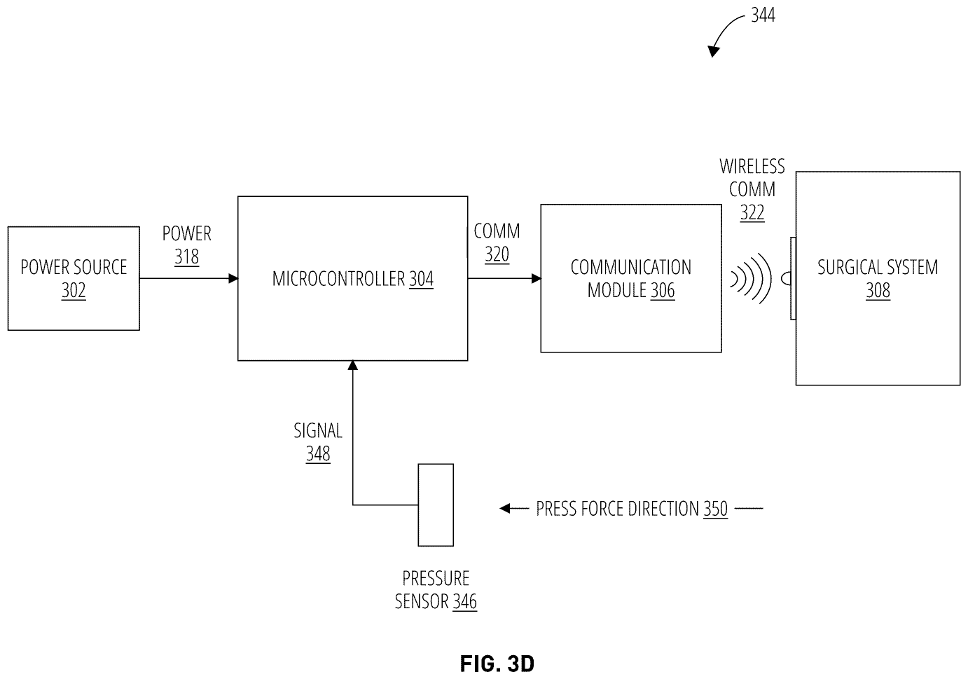

FIGS. 3A-D illustrate various examples of a surgical control system, according to some implementations of the current subject matter. The systems shown in FIGS. 3A-D may be implemented in and/or may incorporate one or more components of the systems 100 (as shown in FIG. 1) and/or 200 (as shown in FIG. 2).

FIG. 3A illustrates an example system 300 that uses a momentary push button 310 for controlling of one or more components of a surgical system, according to some examples of the current subject matter. The system 300 may include a power source 302, a microcontroller or a control processor (terms used interchangeably herewith) 304, a communication module 306, a momentary push button 310, and a trigger 312. The components 302-310 may be communicatively coupled to the surgical system 308 (which may be similar to the system 100 shown in FIG. 1).

The power source 302 may be communicatively coupled to and may provide power 318 the microcontroller 304. The power 318 may be provided using one or more wired and/or wireless connections between the power source 302 and microcontroller 304. Alternatively, or in addition, the power source 302 may be incorporated and/or integrated into the microcontroller 304. The power source 302 may be any type of power source (e.g., an alternating current power source, a direct current power source, a battery, a rechargeable battery, etc.).

The microcontroller 304 may be communicatively coupled to the communication module 306. The microcontroller 304 and the communication module 306 may be coupled using a wired and/or a wireless connection. The microcontroller 304 may use the connection with the communication module 306 to transmit and/or receive one or more communication instructions 320. The instructions 320 may include data, information, etc. containing instructions to the surgical system 308 to actuate, trigger and/or initiate specific action by one or more of its components (e.g., a surgical cutting tool 150 as shown in FIG. 1). The instructions 320 may be received by the communication module 306 and transmitted to the surgical system 308 using a communication link 322. The communication link 322 may be a wireless communication link and/or a wired communication link. In some examples, the communication module 306 may be incorporated and/or integrated into the microcontroller 304, where the microcontroller 304 may then transmit, via the communication link 322, instructions to the surgical system 308 to actuate, trigger, and/or initiate a specific action by one or more of its components (e.g., surgical cutting tool 150).

The microcontroller 304 may also be communicatively coupled to the momentary push button 310. The communicative coupling of the microcontroller 304 and the momentary push button 310 may be using a wireless communication link, which may, for example, include any desired wireless communication protocols, e.g., WiFi, Bluetooth, IOlink, Zigbee, cellular (e.g., 4G LTE, 4G LTE-A, 5G, etc.). In some examples, as discussed herein, one or more custom wireless communication component(s) (e.g., implementing custom-designed software, firmware, hardware, etc.) may be used by the wireless communication module 306 and/or the microcontroller 304 and/or both to communicate with the momentary push button 310, where the custom wireless communication component(s) may implement one or more known wireless communication protocols and/or one or more proprietary communication protocols that may be designed for use with the custom wireless communication component(s). The custom wireless communication component(s) may include, for example, one or more receiver(s), one or more transmitter(s) and/or one or more transceiver(s) that may be communicatively coupled to and/or be integrated with the wireless communication module 306 and/or the microcontroller 304, and/or the momentary push button 310. The momentary push button 310 may include various processing and/or communication circuitry that may be used to generate and/or transmit various wireless instructions (e.g., signal(s) 316) to the microcontroller 304. The instructions may include data, information, etc. that may be used by the microcontroller 304 to determine that a certain action by one or more components (e.g., surgical cutting tool 150) may need to be actuated, triggered, and/or initiated. By way of a non-limiting example, the instructions may include turning on the surgical cutting tool 150, increasing operational speed of the surgical cutting tool 150, decreasing operational speed of the surgical cutting tool 150, activating a graphical user interface of the surgical system 308, initiating tracking by the optical tracking system 140, etc.

In some examples, the momentary push button 310 may be actuated and/or activated using trigger 312, which, in turn, may receive a force in a push direction 314, as shown in FIG. 3A. The force may be temporarily applied, continuously applied, and/or applied in any other desired way. Application of the force in the push direction 314 may cause the trigger 312 to contact the momentary push button 310, thereby closing one or more circuit elements (e.g., a switch) within the momentary push button 310. This may cause one or more of its circuitry to generate one or more actuation instructions (e.g., indication of a desire to turn the surgical cutting tool 150) and one or more of its transmitting circuitry (not shown in FIG. 3A) to transmit the generated actuation instructions (as, for example, signals 316) to the microcontroller 304 for further processing and, eventually, transmitting to the surgical system 308 via the communication module 306.

The momentary push button 310 may be any type of momentary push button. The system 300 may include one or more momentary push buttons 310, whereby each momentary push button 310 may be used by the surgeon using the surgical system 308 to control various aspects of the system 308 prior to, during, and/or after the surgery. Each such button 310 may be communicatively coupled to the communication module 306 and may be activated by the same and/or separate triggers 312. Further, each activation (e.g., using the trigger 312) of the momentary push button 310 (and/or momentary push buttons 310) may result in a specific action to be performed by one or more components of the surgical system 308 that may be determined by the microcontroller 304 and transmitted to the surgical system 308 by the communication module 306. For instance, one press by the trigger 312 on the momentary push button 310 may cause the microcontroller 304 to determine that the surgical cutting tool 150 of the surgical system 308 needs to be turned on. Another press (or type of press, e.g., a double press) by the trigger 312 on the momentary push button 310 may cause the microcontroller 304 to determine that the surgical cutting tool 150 needs to operate at a first operational speed. Yet another press or type of press by the trigger 312 may cause the microcontroller 304 to determine that the surgical cutting tool 150 needs to operate at a second operational speed, which may be faster, slower or same as the first operational speed.

In some examples, the microcontroller 304 may include a sensing circuit and/or component that may be configured to receive signal 316 from the momentary push button 310 and interpret the signals to determine action that may need to be performed by the microcontroller 304. For example, the signals 316 may contain information indicating that the momentary push button 310 was double pressed by the trigger 312. The sensing circuit of the microcontroller 304 may interpret that such double pressing may mean that the operational speed to the surgical cutting tool 150 of the surgical system 308 needs to be increased (e.g., double the speed). As can be understood, any other type of instructions and/or interpretations by the microcontroller 304 and/or any of its components, circuits, etc. are possible.

Once the microcontroller 304 (and/or any of its components, circuits, etc.) has ascertained the meaning of the signals 316, the microcontroller 304 may be configured to generate one or more communication instructions to the communication module 306. The communication module 306 may include an antenna, a transceiver, and/or any other communication circuitry that may be used to receive (via a wireless, wired, and/or any other connection) information, data, etc. from the microcontroller 304 and transmit one or more communication instructions (e.g., signals 322) to the surgical system 308. The communication instructions may include instructions to one or more components (e.g., surgical cutting tool 150) of the surgical system 308 to actuate, trigger, and/or initiate one or more actions. For example, a communication instructions may include turning on the surgical cutting tool 150, increasing and/or decreasing its operational speed, initiating tracking by optical tracking system 140 of the surgical system 308, and/or any other action and/or any combination thereof. The surgical system 308 may include one or more transceivers that may be configured to receive the communication instructions and determine the intended actions by one or more of its components.

FIG. 3B illustrates an example system 324 that uses a linear potentiometer 326 for controlling of one or more components of a surgical system, according to some examples of the current subject matter. The system 324 is similar to the system 300 shown in FIG. 3A, in that it, similarly, may include the power source 302, the microcontroller 304, and the communication module 306 and may be configured to communicate with the surgical system 308.

As stated above, in order to provide actuation instructions to the microcontroller 304, the system 324 may implement the linear potentiometer 326 and a trigger 328. The linear potentiometer 326 may be a position sensor that may measure a displacement along an axis (e.g., up and down, left and right, etc.), which may result upon application of a press force in a direction 332. The linear potentiometer 326 may be rod actuated, such as, for example, using the trigger 328 to which the press force is applied in the direction 332. It may also include an internal slider, a wiper carrier, etc. that may be configured to translate along the axis once the pressure from the trigger 328 is detected. Once a predetermined linear displacement is detected by the circuitry of the linear potentiometer 326, the linear potentiometer 326 may be configured to generate one or more actuation instructions (similar to the momentary push button example discussed in connection with FIG. 3A).

The actuation instructions may be transmitted to the microcontroller 304 using one or more signals 330. Upon receiving the signal(s) 330, the microcontroller 304 and/or its sensing circuit and/or component may be configured to interpret the signals signal 330 and determine action that may need to be performed by the microcontroller 304. For example, the signals 330 may contain information indicating that the rod of the linear potentiometer 326 may have been translated a first predetermined distance (e.g., 1 millimeter). Such translation may be indicative of the surgeon's desire to turn on one or more components of the surgical system 308 (e.g., surgical cutting tool 150). Upon receiving of further signals 330, the microcontroller 304 may interpret them to determine that a further translation (e.g., 2 millimeters) has occurred within the linear potentiometer 326, which may mean that the operational speed to the surgical cutting tool 150 needs to be increased. As can be understood, any other type of instructions and/or interpretations by the microcontroller 304 and/or any of its components, circuits, etc. in response to signals 330 are possible.

As discussed above in connection with FIG. 3A, the signals 330, as interpreted by the microcontroller 304, may then be passed on, as one or more communication instructions, to the communication module 306 for transmission to the surgical system 308. In some examples, the communication instructions may be transmitted dynamically (e.g., based on the timing of receipt/interpretation of the signals 330), periodically, continuously, and/or using any other schedule. As discussed in connection with FIG. 3A, one or more custom wireless communication components and/or custom wireless communication protocols may be used for transmission, receiving, etc. of communication instructions.

FIG. 3C illustrates an example system 334 that uses a pressure sensor 336 for controlling of one or more components of a surgical system, according to some examples of the current subject matter. The system 334 may be similar to the systems 300 and 324 shown in FIGS. 3A and 3B, respectively. It may, likewise, include the power source 302, the microcontroller 304, and the communication module 306 and may be configured to communicate with the surgical system 308.

In this example, the system 334 may implement the pressure sensor 336 and a trigger 338. The pressure sensor 336 may be a transducer that may convert an input mechanical pressure (e.g., as a result of application of press force in a direction 342 to the trigger 338) into an electrical output signal (e.g., pressure sensor defined). The output electrical system may be used to generate actuation instructions by the pressure sensor circuitry. Upon detecting a predetermined pressure by the pressure sensor 336, the pressure sensor 336 may generate one or more actuation instructions (similar to the instructions discussed in connection with FIGS. 3A and 3B above).

The pressure sensor 336 may then transmit generated actuation instructions to the microcontroller 304 using one or more signals 340. The microcontroller 304 and/or its sensing circuit/component may interpret the signals 340 and determine any action(s) that may need to be executed by the microcontroller 304. For instance, the signals 340 may include information indicating that the pressure sensor 336 detected a first pressure (e.g., a slight tap by the surgeon's finger), which may indicate the surgeon's desire to turn on surgical cutting tool 150 of the surgical system 308. Upon receiving of further signals 340, the microcontroller 304 may interpret them to determine that a further pressure (e.g., a firmer press than a slight finger tap) has been detected by the pressure sensor 336, which may be indicative of the surgeon's desire to increase operational speed to the surgical cutting tool 150. As can be understood, any other type of instructions and/or interpretations by the microcontroller 304 and/or any of its components, circuits, etc. in response to signals 340 are possible. Similar to the discussion with regard to FIGS. 3A-B, the signals 340, as interpreted by the microcontroller 304, may then be passed on, as one or more communication instructions, to the communication module 306 for transmission to the surgical system 308. Again, as discussed in connection with FIGS. 3A-B, custom wireless communication component(s) and/or custom wireless communication protocol(s) may be used for transmission, receiving, etc. of communication instructions.

FIG. 3D illustrates an example system 344 that uses a pressure sensor 346 for controlling of one or more components of a surgical system, according to some examples of the current subject matter. Similar to FIGS. 3A-C, the system 344 may likewise include the power source 302, the microcontroller 304, and the communication module 306 and may be configured to communicate with the surgical system 308.

However, the system 344, unlike system 334 shown in FIG. 3C, does not include a trigger, and instead, its pressure sensor 346 may be configured to receive pressure (as applied by a press force in direction 350) directly. Thus, upon detecting a predetermined pressure by the pressure sensor 346, the sensor 346 may generate one or more actuation instructions (similar to the instructions discussed in connection with FIGS. 3A-C above) and then, transmit them to the microcontroller 304 using one or more signals 348 for interpretation. The microcontroller 304 and/or its sensing circuit/component may then determine action(s) to be executed by the microcontroller 304. Examples of actions are discussed above with regard to FIGS. 3A-C.

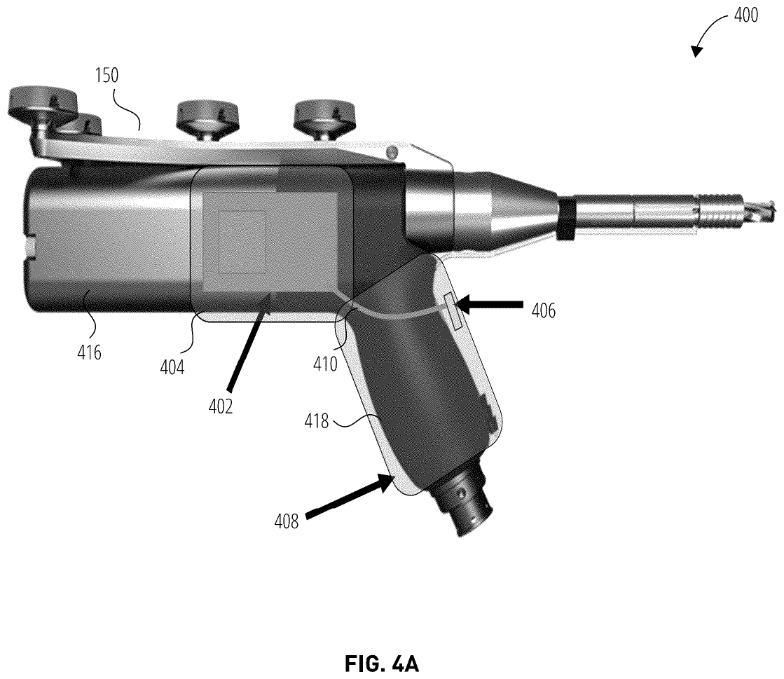

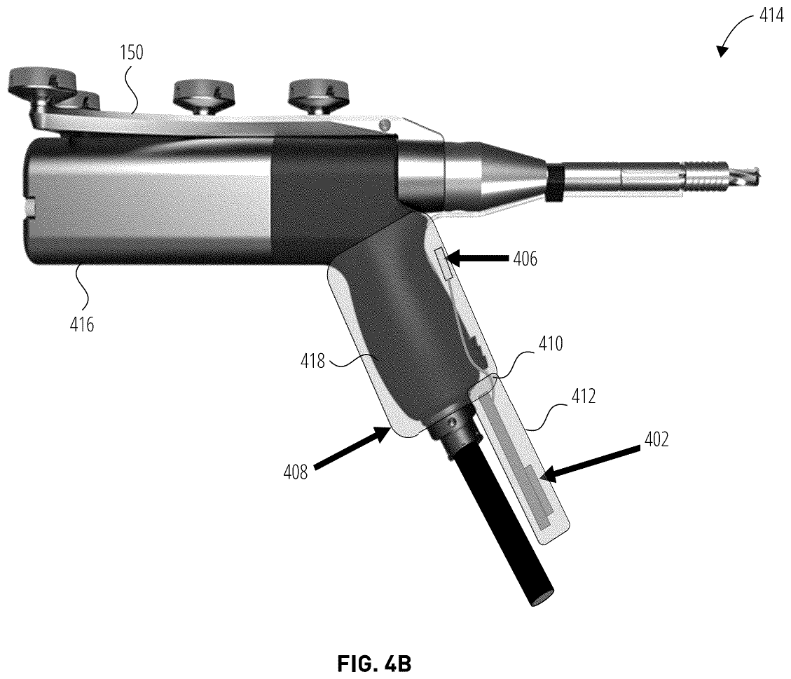

FIGS. 4A and 4B illustrate surgical systems 400 and 414, respectively, according to some examples of the current subject matter. The systems 400, 414 may be used for controlling operation of the surgical cutting tool 150, for example. Referring to FIG. 4A, the surgical system 400 may include surgical cutting tool 150, microprocessor and transceiver circuit 402 embedded into a molding 404, and a sensor 406 embedded into a molding 408. The microprocessor and transceiver circuit 402 and the sensor 406 may be communicatively coupled using a communication connection 410. The connection 410 may be a wired and/or a wireless connection. The molding 404 may be coupled to the body 416 of the surgical cutting tool 150 and the molding 408 may be coupled to the handle 418 of the surgical cutting tool 150. In some examples, one or more of the microprocessor and transceiver circuit 402, the sensor 406, connection 410, and/or the moldings 404, 408 (412 as shown in FIG. 4B) may be disposable, which may make it easier to replace, such as, for example, when an upgrade is needed, one or more of them malfunction, sterilization of various components of the surgical system and/or control system is needed, and/or for any other reasons. Alternatively, or in addition, one or more of these components (e.g., the microprocessor and transceiver circuit 402, the sensor 406, one or more parts of and/or entirety of the connection 410, etc.) may be integrated into the tool 150 (e.g., with and/or without use of the moldings).

The microprocessor and transceiver circuit 402 may include a microprocessor circuity and a transceiver circuitry. The microprocessor circuitry may be similar to the microcontroller 304 shown in FIGS. 3A-D and the transceiver circuitry may be similar to the communication module 306. The sensor 406 may be similar to one or more of the momentary push button 310 (shown in FIG. 3A), linear potentiometer 326 (shown in FIG. 3B), pressure sensor 336 (shown in FIG. 3C), and/or pressure sensor 346 (shown in FIG. 3D). The connection 410 may be configured to be used for transmission of signals 316, 330, 340, and/or 348 that may be contain actuation instructions.

The microprocessor and transceiver circuit 402 may be configured to receive actuation instructions from the sensor 406 and process them to generate one or more communication instructions to the surgical cutting tool 150 to perform a desired action. The transceiver circuitry (which may include an antenna, a wireless module, etc.) of the microprocessor and transceiver circuit 402 may be configured to transmit such communication instructions to the surgical cutting tool 150 causing it to perform one or more desired actions in accordance with the communication instructions.

In some examples, the molding 404 may be coupled and/or attached to the body 416 of the surgical cutting tool 150. The molding 408 may be coupled and/or attached to the handle 418 of the surgical cutting tool 150. The moldings 404, 408 may be attached to the surgical cutting tool 150 using any desired mechanisms, e.g., gluing, welding, heat molding, Velcro™, etc. The moldings 404 and/or 408 may be silicone moldings and/or any other type of moldings. As shown in FIG. 4A, separate moldings may integrate microprocessor and transceiver circuit 402 and the sensor 406. Alternatively, or in addition, a single molding may integrate the microprocessor and transceiver circuit 402 and the sensor 406. Further, the microprocessor circuitry and the transceiver circuity of the microprocessor and transceiver circuit 402 may be separate and may be integrated into their own respective moldings. The moldings 404 and/or 408 (along with corresponding microprocessor and transceiver circuit 402 and/or sensor 406) may be positioned in any desired location on the surgical cutting tool 150 and/or on any other component of the system 100 shown in FIG. 1.

FIG. 4B illustrates the surgical system 414, according to some examples of the current subject matter. The system 414 may be similar to the system 400 shown in FIG. 4A in that it includes the surgical cutting tool 150, the microprocessor and transceiver circuit 402, and the sensor 406 communicatively coupled to the circuit 402 using connection 410.

As shown in FIG. 4B, the microprocessor and transceiver circuit 402 may be integrated into a molding 412 that may be coupled to the molding 408. The molding 408 may, in turn, be coupled to the handle 418 of the surgical cutting tool 150. Coupling of the moldings 408 and 412 may be using any desired methods, e.g., gluing, welding, heat-molding, Velcro™, etc.

As can be understood, the current subject matter is not limited to surgical cutting tools (e.g., surgical saws, etc.), and may likewise be applicable to any other surgical tools, e.g., sagittal saws, surgical impactors, surgical reamers, surgical drills, multi-functional surgical systems (e.g., surgical drill and saw systems, etc.), and/or any other tools.

Further, the current subject matter may be configured to implement one and/or multiple sensors 406 that may be positioned in any desired location. This may allow users to actuate the surgical tool from any location, such as, for example, but not limited to, when the surgical tool is positioned in any desired position (e.g., right side up, upside down, at an angle (e.g., with respect to the surgical site)), at a particular height above the surgical site (e.g., such as, in the case of surgical reamers). The sensors may be configured to communicate with one another to prevent unnecessary actuations and/or interruptions of operations of the surgical tool. For example, upon actuation of one sensor (e.g., user pressing that sensor), one or more of the other sensors may be disabled to prevent cross-communications from sensors and, hence, undesired operation of the surgical tool.

Multiple transceiver circuits may likewise be used and may be configured to communicate with one another and/or the circuit 402. Further, multiple connections 410 among the transceiver circuit(s) and/or the circuit 402 may be formed. Likewise, single and/or multiple moldings 408, 412 may be used to retain the sensors, transceivers, connections, and/or any other electronic components. The moldings may be surgical tool specific, user-specific, and/or be generic allowing the moldings to be used with any type of surgical tool. Similarly, the sensors and/or transceivers may be configured to be used with a particular surgical tool and/or a particular user. Alternatively, or in addition, the sensors and/or transceivers may be designed to be used with any surgical tool and/or any user.

The sensors, transceivers, etc. may also be activatable/de-activatable (e.g., activated for the purposes of detecting user's action (e.g., pressing), etc.) based on a particular position, orientation, location, etc. of the surgical tool. For example, a first sensor and/or transceiver, etc. positioned on (e.g., through use of one or more moldings 408, 412) one side of a handle of the surgical tool may be activated for detection of user's action, while a second sensor and/or transceiver positioned on (e.g., through use of one or more moldings 408, 412) on another side of the handle of the surgical tool may be deactivated when the surgical tool is positioned right side up. However, when the surgical tool is positioned upside down, the first sensor and/or transceiver may be deactivated while the second sensor and/or transceiver may be activated. This may allow the user of the surgical tool to use different hands and/or different fingers (e.g., thumb, index finger, etc. that may be positioned in a natural position on the surgical tool) to actuate the surgical tool. Various surgical tool orientation, position, location, etc. detection components (e.g., accelerometers, coordinate sensors, etc.) may be used to determine surgical tool's orientation, position, location, etc. and use such determined data, to activate and/or deactivate one or more sensors and/or transceivers for the purposes of actuation of the surgical tool.

In some examples, the sensors, transceivers, etc. may be positioned anywhere on the surgical tool. For example, in case of surgical drills, surgical impactors, etc., the sensors, transceivers, etc. may be positioned on the handle of the surgical tool, on the body of the surgical tool, on the nozzle of the surgical tool, on an attachable handle (e.g., an impact handle that may be attached to the body of surgical tool, etc.), etc. In case of surgical reamers, for instance, the sensors, transceivers, etc. may be positioned on the handle, body, attachable handle, and/or remotely from the surgical tool, which may allow its users greater accessibility and operability. As can be understood, any positioning of moldings, sensors, transceivers, connections, circuits, etc. and/or use thereof in connection with any desired surgical tool are possible.

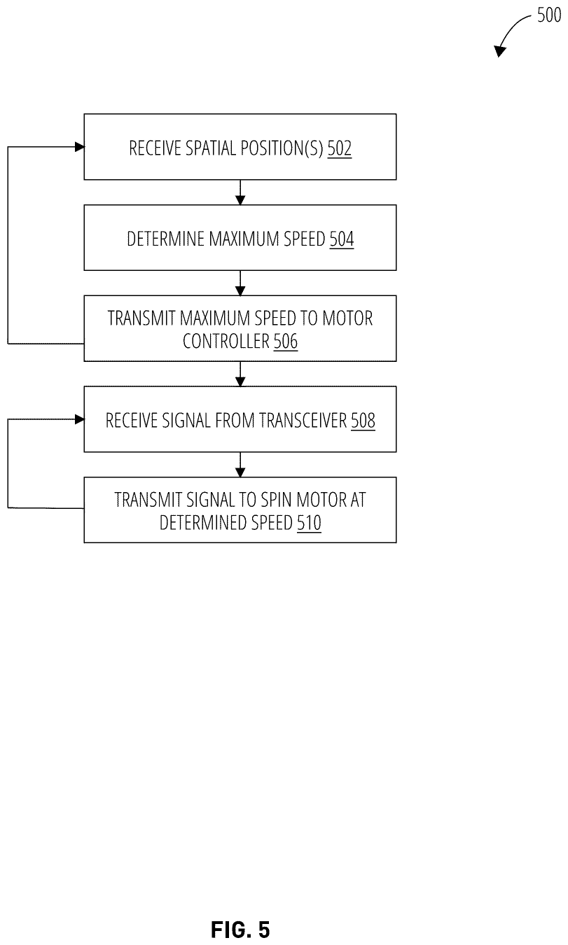

FIG. 5 illustrates a process 500 for controlling operation of the surgical cutting tool 150, according to some examples of the current subject matter. The process 500 may be executed using systems shown in FIGS. 3A-D and/or FIGS. 4A-B. In particular, the microprocessor and transceiver circuit 402 and/or one or more processors of the surgical cutting tool 150 may be configured to execute one or more operations of the process 500.

At 502, one or more spatial positions related to the positioning the patient, the surgical cutting tool 150, and/or any other component of the system 100 shown in FIG. 1 may be received. The spatial positions may be received from optical tracking system 140 of the system 100, which may include one or more cameras, sensors, etc. The spatial positions may be represented as one or more coordinates, images, graphs, etc. Such positions may be determined by one or more processors of the system 100, including a processor that may be integrated into the surgical cutting tool 150.

At 504, one or more operating speeds, including a maximum operating speed, of a motor of the surgical cutting tool 150 may be determined. The operating speeds may be determined by one or more processors of the system 100, including the processor of the surgical cutting tool 150. The speeds may then be transmitted to a motor controller that may trigger operation of the motor of the surgical cutting tool 150, at 506. One or more operations 502-506 may be iteratively performed by one or more processors of the system 100 prior to, during, and/or after the surgical procedure.

At 508, one or more processors of the system 100 may be configured to receive one or more signals that may include one or more communication instructions (as discussed in connection with FIGS. 3A-D above). The signals (e.g., signals 322) may be transmitted from communication module 306, e.g., a transceiver, an antenna, etc., (as shown in FIGS. 3A-D) and in response to receiving of one or more actuation instructions from one or more sensors 406 (as shown in FIGS. 4A-B). As discussed herein, the sensors 406 may include the momentary push button 310, the linear potentiometer 326, the pressure sensor 336, the pressure sensor 346, and/or any other type of sensor. The signals may then be transmitted, at 510 to one or more processors of the system 100, including the surgical cutting tool 150, and may trigger operation of the motor of the surgical cutting tool 150 at one or more speeds determined, at 502-506. The signals representing communication instructions may be received dynamically, periodically, and/or on continuous basis. This may allow variable control of operating speed(s) of the surgical cutting tool 150 during a surgical procedure.

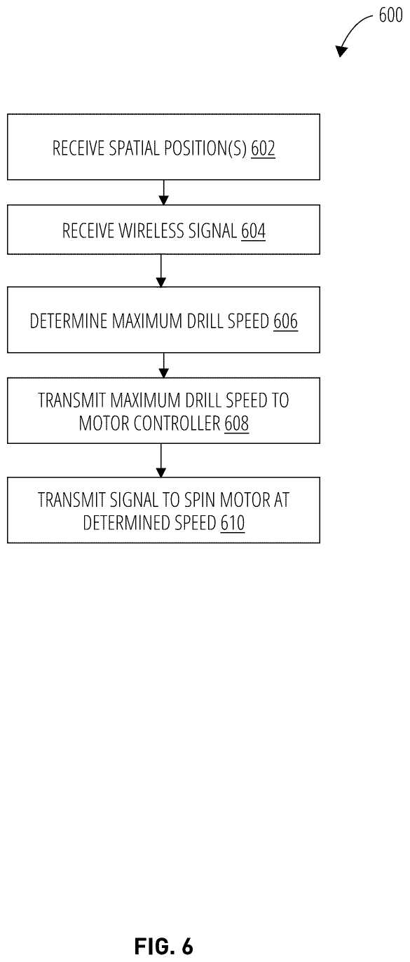

FIG. 6 illustrates another process 600 for controlling operation of the surgical cutting tool 150, according to some examples of the current subject matter. The process 600 may likewise be executed using systems shown in FIGS. 3A-D and/or FIGS. 4A-B. In particular, the microprocessor and transceiver circuit 402 and/or one or more processors of the surgical cutting tool 150 may be configured to execute one or more operations of the process 600.

At 602, one or more spatial positions related to the positioning the patient, the surgical cutting tool 150, and/or any other component of the system 100 shown in FIG. 1 may be received from the optical tracking system 140. At 604, one or more processors of the system 100 may receive one or more signals containing one or more communication instructions, where the signals (e.g., signals 322) may be transmitted from the communication module 306, e.g., a transceiver, an antenna, etc., (as shown in FIGS. 3A-D) and in response to one or more actuation instructions from one or more sensors 406 (as shown in FIGS. 4A-B).

Using the spatial positions and in response to receiving signals representing the communication instruction(s), one or more processors of the system 100 may determine one or more operating speeds, including a maximum operating speed, of a motor of the surgical cutting tool 150, at 606. The determined operating speeds and/or the communication instructions may be transmitted to motor controller, at 608, which may trigger operation of the motor of the surgical cutting tool 150, at 610.

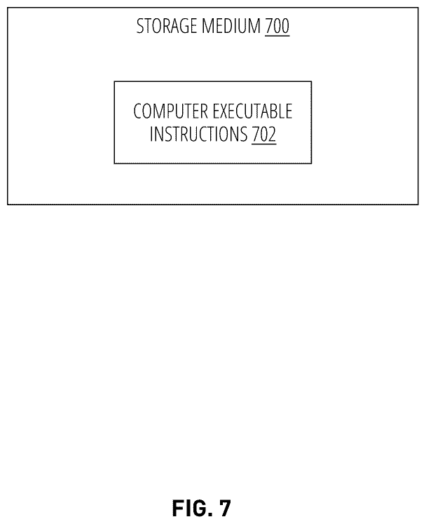

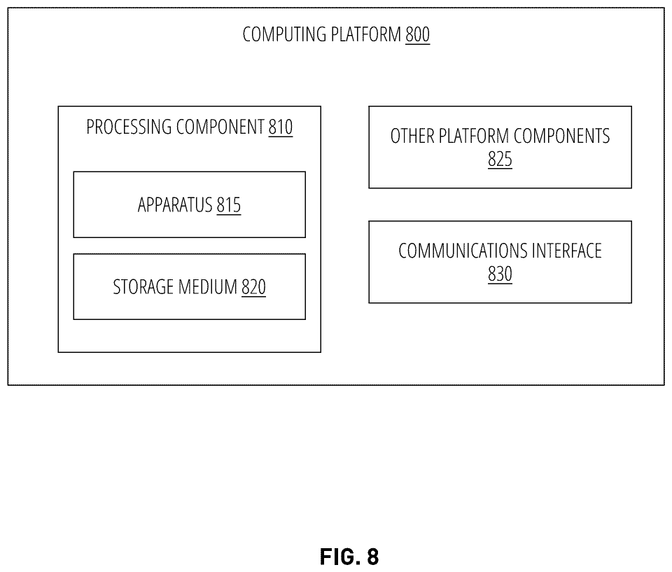

FIG. 7 and FIG. 8 illustrate example implementations of a storage medium and computing platform for an orthopedic surgical instrument or a surgical system in accordance with one or more features of the present disclosure. FIG. 7 illustrates an example of a storage medium 700 to store system logic. Storage medium 700 may include an article of manufacture. In some examples, storage medium 700 may include any non-transitory computer readable medium or machine readable medium, such as an optical, magnetic or semiconductor storage. Storage medium 700 may store various types of computer executable instructions 702, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 8 illustrates an example computing platform 800. In some examples, as shown in FIG. 8, the computing platform 800 may include a processing component 810, other platform components 825 or a communications interface 830. According to some examples, computing platform 800 may be implemented in a computing device such as a server in a system such as a data center or server farm that supports a manager or controller for managing configurable computing resources as mentioned above. Further, the communications interface 830 may include a wake-up radio (WUR) and may be capable of waking up a main radio of the computing platform 800.

According to some examples, processing component 810 may execute processing operations or logic for apparatus 815 described herein such as the microcontroller 304, communication module 306, and/or one or more processors of the surgical system 100. Processing component 810 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 820, may include software components, programs, applications, computer programs, application programs, device drivers, system programs, soft ware development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 825 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 830 may include logic and/or features to support a communication interface. For these examples, communications interface 830 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 800 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 800 described herein, may be included or omitted in various implementations of computing platform 800, as suitably desired.

The components and features of computing platform 800 may be implemented using any combination of discrete circuitry, ASICs, logic gates and/or single chip architectures. Further, the features of computing platform 800 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 800 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation”, “embodiments”, and/or “examples” (terms used interchangeably herein) (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this detailed description by this reference, with each claim standing on its own as a separate implementation of the present disclosure.

What is claimed is:

1. A system, comprising:
a wireless actuator, upon actuation, configured to generate and transmit one or more actuation instructions; and
a control processor communicatively coupled to the wireless actuator;
the control processor is configured to
receive the one or more actuation instructions from the wireless actuator;
generate, using the one or more actuation instructions, one or more communication instructions for triggering actuation of at least one operation of one or more surgical components of a surgical system communicatively coupled to the control processor; and
transmit, via an antenna communicatively coupled to the control processor, the one or more communication instructions to the surgical system.

2. The system of claim 1, wherein
upon a first actuation, the wireless actuator is configured to generate and transmit a first actuation instruction in the one or more actuation instructions to the control processor causing the control processor to generate and transmit a first communication instruction in the one or more communication instructions triggering actuation of a first operation of the one or more surgical components;
upon a second actuation, the wireless actuator is configured to generate and transmit a second actuation instruction in the one or more actuation instructions to the control processor causing the control processor to generate and transmit a second communication instruction in the one or more communication instructions triggering actuation of a second operation of the one or more surgical components, the second operation being subsequent to the first operation.

3. The system of claim 2, wherein the second operation is different from the first operation.

4. The system of claim 2, wherein the second operation is the same as the first operation.

5. The system of claim 2, wherein the first operation is a surgical cutting operation performed by the one or more components of the surgical system at a first speed and a second operation is a surgical cutting operation performed by the one or more components of the surgical system at a second speed.

6. The system of claim 5, wherein the second speed is faster, same or slower than the first speed.

7. The system of claim 5, wherein at least one of the first speed and the second speed is at least one of: a variable speed, a constant speed, and any combination thereof.

8. The system of claim 1, wherein the wireless actuator includes a momentary push button and a trigger.

9. The system of claim 8, wherein, upon pressing the trigger, the trigger is configured to apply pressure to the momentary push button causing the momentary push button to generate and transmit the one or more actuation instructions.

10. The system of claim 1, wherein the wireless actuator includes a linear potentiometer and a trigger.

11. The system of claim 10, wherein, upon pressing the trigger, the linear potentiometer is configured to determine its linear displacement causing the linear potentiometer to generate and transmit the one or more actuation instructions.

12. The system of claim 1, wherein the wireless actuator includes a pressure sensor and a trigger.

13. The system of claim 12, wherein, upon pressing the trigger, the pressure sensor is configured to detect a predetermined force applied to it by the trigger causing the pressure sensor to generate and transmit the one or more actuation instructions.

14. The system of claim 1, wherein the wireless actuator includes a pressure sensor.

15. The system of claim 14, wherein, upon applying a force to the pressure sensor, the pressure sensor is configured to detect the force causing the pressure sensor to generate and transmit the one or more actuation instructions.

16. The system of claim 1, wherein the control processor includes a sensing circuit configured to
receive the one or more actuation instructions;
interpret the one or more actuation instructions; and
generate and transmit, via the antenna communicatively coupled to the control processor, the one or more communication instructions to the surgical system.

17. The system of claim 1, wherein at least one of the control processor, the antenna, and the wireless actuator are disposed in the one or more surgical components of the surgical system.

18. A method, comprising:
receiving, using at least one processor, one or more actuation instructions from a wireless actuator communicatively coupled to the at least one processor, wherein the wireless actuator, upon actuation, is configured to generate and transmit the one or more actuation instructions to the at least one processor;
generating, using the at least one processor, using the one or more actuation instructions, one or more communication instructions for triggering actuation of at least one operation of one or more surgical components of a surgical system communicatively coupled to the at least one processor; and
transmitting, using the at least one processor, via an antenna communicatively coupled to the at least one processor, the one or more communication instructions to the surgical system.

19. The method of claim 18, wherein
upon a first actuation, the wireless actuator is configured to generate and transmit a first actuation instruction in the one or more actuation instructions to the at least one processor causing the at least one processor to generate and transmit a first communication instruction in the one or more communication instructions triggering actuation of a first operation of the one or more surgical components;
upon a second actuation, the wireless actuator is configured to generate and transmit a second actuation instruction in the one or more actuation instructions to the at least one processor causing the at least one processor to generate and transmit a second communication instruction in the one or more communication instructions triggering actuation of a second operation of the one or more surgical components, the second operation being subsequent to the first operation.

20. The method of claim 19. wherein the first operation is a surgical cutting operation performed by the one or more components of the surgical system at a first speed and a second operation is a surgical cutting operation performed by the one or more components of the surgical system at a second speed.
